(19) United States
(12) Patent Application Publication
(10) Pub. No.: US 2016/0279246 A1
(43) Pub. Date: Sep. 29, 2016

(54) METHODS AND SYSTEMS FOR CONTINUOUS HETEROGENEOUS CRYSTALLIZATION

(71) Applicant: Massachusetts Institute of Technology, Cambridge, MA (US)

(72) Inventors: Bernhardt Levy Trout, Lexington, MA (US); Allan Stuart Myerson, Boston, MA (US); Siva Rama Krishna Perala, Malden, MA (US); Christopher James Testa, Winthrop, MA (US); Keith Dale Jensen, Arlington, MA (US)

(73) Assignee: Massachusetts Institute of Technology, Cambridge, MA (US)

(21) Appl. No.: 15/054,956

(22) Filed: Feb. 26, 2016

Related U.S. Application Data
(60) Provisional application No. 62/137,143, filed on Mar. 23, 2015.
(60) Provisional application No. 62/126,383, filed on Feb. 27, 2015.

(51) Int. Cl. A61K 47/26; A61K 31/167

(52) U.S. Cl. CPC A61K 47/26; A61K 31/167

(57) ABSTRACT

Methods of heterogeneous crystallization and related systems are provided. In some embodiments, a method comprises crystallizing an agent in a suspension comprising a heteronucleant and the dissolved agent. Crystallization may occur on the surface of the heteronucleant with little or no bulk crystallization and/or secondary nucleation. In some embodiments, a crystallizer may be configured to inhibit secondary nucleation and/or bulk crystallization, for example, by reducing the formation of free crystals that may serve as nucleation surfaces while allowing for adequate mass and heat transfer. In some such embodiments, the crystallizer may be designed to flow (e.g., continuously) a suspension comprising a heteronucleant and an agent in a fluidized state. The methods and systems of the present invention may be used in a wide variety of applications, including the crystallization of pharmaceutically active agents.

### RELATED APPLICATIONS

This application claims the benefit of U.S. Provisional Patent Application Ser. No. 62/137,143, filed Mar. 23, 2015, entitled “Methods and Systems for Continuous Heterogeneous Crystallization,” and U.S. Provisional Patent Application Ser. No. 62/126,383, filed Feb. 27, 2015, entitled “Apparatus and Method for the Crystallization of Active Pharmaceutical Ingredients on Crystalline Excipients,” both of which are incorporated herein by reference in its entirety.

### TECHNICAL FIELD

Methods of heterogeneous crystallization and related systems are generally described.

### BACKGROUND

In many areas of science and technology, such as the production of pharmaceuticals, semiconductors, and optics, as well as the formation of biominerals, the ability to control crystallization is desired. As will be known to those of ordinary skill in the art, nucleation is generally a critical step in controlling the crystallization process. While many studies have been conducted regarding controlling crystallization of small organic molecules, crystallization is a complex and not well understood process. In addition, generally, small organic molecules may be crystallized in a variety of crystal patterns, and it is difficult, if not impossible, to predict under which conditions, a small organic molecule will crystallize.

Accordingly, improved methods and systems are needed.

### SUMMARY

Methods of heterogeneous crystallization and related systems are provided. The subject matter of the present invention involves, in some cases, interrelated products, alternative solutions to a particular problem, and/or a plurality of different uses of one or more systems and/or articles.

In one set of embodiments, methods are provided. In one embodiment, a method comprises flowing a suspension comprising an excipient and a dissolved pharmaceutically active agent and while the suspension is flowing, crystallizing at least a portion of the pharmaceutically active agent on at least a portion of a surface of the excipient.

In another embodiment, a method comprises crystallizing a pharmaceutically active agent in a suspension comprising the pharmaceutically active agent, an excipient, and a solvent to form a plurality of pharmaceutically active agent crystals, wherein greater than or equal to about 80% of the pharmaceutically active agent crystals are in contact with a surface of the excipient.

In one embodiment, a method comprises agitating a suspension comprising an excipient and a dissolved pharmaceutically active agent in a crystallizer and while agitating, crystallizing the pharmaceutically active agent on at least a portion of a surface of the excipient to form pharmaceutically active agent crystals, wherein, during the agitation, fewer than or equal to about 20% of the pharmaceutically active crystals that collide with a solid surface fracture as a result of the collisions.

In another embodiment, a method comprises agitating a suspension comprising an excipient and a dissolved pharmaceutically active agent in a crystallizer; and while agitating, crystallizing the pharmaceutically active agent on at least a portion of a surface of the excipient to form pharmaceutically active agent crystals, wherein, during the agitation, fewer than or equal to about 20% of pharmaceutically active crystals collide with a solid surface within the crystallizer with a force of greater than 1.2 N.

In one embodiment, a method comprises crystallizing a pharmaceutically active agent in a suspension comprising the pharmaceutically active agent, an excipient, and a solvent to form a plurality of pharmaceutically active agent crystals, wherein fewer than to about 20% of the pharmaceutically active agent crystals are formed via a bulk crystallization process and/or secondary nucleation process.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 shows a schematic of a crystallizer, according to one set of embodiments;

FIG. 2A-2B show a schematic of a crystallizer, according to certain embodiments;

FIG. 3 shows a graph of concentration of acetaminophen versus time measured using FTIR, according to one set of embodiment;

FIG. 4 shows an XPRD analysis of D-mannitol and acetaminophen as well as the product crystal, according to certain embodiments;

FIG. 5 shows a microscope image showing acetaminophen crystals over D-mannitol surface, according to one set of embodiments; and

FIG. 6 shows tablets made out of the product from a crystallizer, according to certain embodiments;

FIG. 7 shows a schematic of a crystallizer, according to one set of embodiments; and

FIG. 8 shows microscope images of (top) D-mannitol seeds, (middle) a composition from the crystallizer after a certain operation time having acetaminophen crystals on D-mannitol surface, and (bottom) a composition from the crystallizer at a later operation time having acetaminophen crystals on D-mannitol surface, according to one set of embodiments.

### DETAILED DESCRIPTION

Methods of heterogeneous crystallization and related systems are provided. In some embodiments, a method comprises crystallizing an agent (e.g., pharmaceutically active agent) in a suspension comprising a heteronucleant (e.g., excipient) and the dissolved agent. Crystallization may occur on the surface of the heteronucleant with little or no bulk crystallization and/or secondary nucleation (i.e., nucleation induced by the presence of crystals of the substance that is being crystallized). In some embodiments, a crystallizer may be configured to inhibit secondary nucleation and/or bulk crystallization, for example, by reducing (e.g., minimizing or eliminating) the formation of free crystals (i.e., crystals that are not directly bound, e.g., physically to the heteronucleant) that may serve as nucleation surfaces. In some such embodiments, the crystallizer may be designed to flow (e.g., continuously) a suspension comprising a heteronucleant and a dissolved agent in a fluidized state. The methods and systems of the present invention may be used in a wide variety of applications, including the crystallization of pharmaceutically active agents (e.g., active pharmaceutical ingredient).

Many commercial crystalline materials are formed via industrial crystallization processes. For example, crystallization is a common technique used to purify pharmaceutically active agents in pharmaceutical manufacturing processes. Generally, after the pharmaceutically active agent has been crystallized, the crystals are granulated and blended with excipients in a series of solid state operations. The granulation and blending steps may be problematic, for example, as the steps may be plagued by poor process control and/or final product uniformity, and/or the process parameters may be very sensitive to the properties of the specific type of pharmaceutically active agent. In addition, bulk crystallization of pharmaceutically active agents may not always provide a single type of polymorph of the pharmaceutically active agent and/or changes over time during a manufacturing process can cause different types of polymorphs to form. For example, a slight change in temperature may cause a pharmaceutically active agent to crystallize in a phase different than the desired phase. In addition, granulating and/or blending steps may induce changes in the crystal phase of the pharmaceutically active agent. Moreover, secondary nucleation may lead to poor process control ability and/or final product uniformity.

It has been discovered, within the context of certain embodiments of the present invention, that a controlled heterogeneous crystallization process with little or no bulk crystallization and/or secondary nucleation may be achieved using a suspension comprising a dissolved agent (e.g., a dissolved pharmaceutically active agent) and an heteronucleant (e.g., excipient). The suspension may be formed in and/or added to a crystallizer configured to operate under conditions that maintain appropriate mass and heat transfer without employing harsh mixing techniques (e.g., certain propeller induced mixing) that may form a significant amount of free crystals (e.g., via fracture of crystals bound to the heteronucleant due to collision with one or more solid surfaces within the crystallizer) that may serve as nucleation surfaces. It should be understood that as used herein, suspension has its ordinary meaning in the art. In some embodiments, a suspension refers to a heterogeneous mixture containing solid particles (e.g., heteronucleant, pharmaceutically active agent crystals), one or more solvents in which the solid particles are substantially insoluble, and/or one or more materials (e.g., pharmaceutically active agent) dissolved in the solvent.

In some embodiments, the crystallizer may operate under conditions that fluidize the suspension comprising the agent and heteronucleant. The fluidized suspension may act as fluidized bed with the heteronucleants, and accordingly bound crystals, serving as the particulate matter. In some embodiments, the fluidized bed may be flowed within a closed loop until a desired point is reached (e.g., circulation time, crystal size). For example, at least a portion of the suspension may be removed from the closed loop after a certain time and flowed to a downstream filtration unit. In some instances, the crystallizer may be operated in a continuous manner. As another example, at least a portion of the heteronucleant (e.g., excipient) in the suspension may be removed based on the mass of the agent crystal bound to the heteronucleant using separation methods that depend on density. The density of the heteronucleant may depend, at least in part, on the mass of agent crystals bound to the heteronucleant. In such cases, agent crystals having, e.g., a relatively large mass may be removed while leaving smaller crystals in the suspension for continued crystal growth. In some cases, removal of the particles may be based on their size. Particles above a certain size may be removed from the fluidization chamber. Smaller particles may be retained or returned to the column for continued crystal growth.

Regardless of how the agent crystals (e.g., pharmaceutically active agent bound to a surface of an excipient) are removed from the crystallizer, the crystals may be suitable for use in a product either immediately after removal from the crystallizer or after a purification step (e.g., filtration and/or drying). For example, pharmaceutically active agent crystals directly physically bound to the surface of an excipient may be removed from the crystallizer via filtration, dried, and used directly in a pharmaceutical composition (e.g., bound crystals may be compressed to form a tablet and/or encapsulated). In some such embodiments, the methods and systems as described may aid in reducing and/or eliminating typical pharmaceutical formulation processing steps such as granulation and/or blending as well as increase the final product's uniformity. Thus, the crystallization and/or nucleation techniques described herein can provide a greater ability to control the uniformity of the crystals, control the crystal phase of the pharmaceutically active agent, and/or reduce or eliminate processing steps which may result in changes in the crystal phase that may occur during these steps.

In some embodiments, a pharmaceutically active agent that nucleates on a surface of the heteronucleant may be bound to the heteronucleant via a non-covalent bond and/or a physical interaction. For example, the pharmaceutically active agent may be bound via a surface of the heteronucleant via a non-covalent bond, such as an ionic bond, a hydrogen bond (e.g., between hydroxyl, amine, carboxyl, thiol, and/or similar functional groups), a dative bond (e.g., complexation or chelation between metal ions and monodentate or multidentate ligands), Van der Waals interactions, or the like.

Exemplary crystallization methods and systems will now be described in more detail. It should be understood that while many of the following embodiments described herein discuss an agent being a pharmaceutically active agent and a heteronucleant being an excipient, this is by no way limiting and other agents and heteronucleants may be used.

As described herein, in some embodiments, a method for selective heterogeneous crystallization of an agent on a heteronucleant may comprise flowing a suspension comprising the heteronucleant and a dissolved agent (e.g., in a crystallizer, in a continuous flow crystallizer) and crystallizing at least a portion of the agent on at least a portion of a surface of the heteronucleant during the flow process. In some embodiments, the agent may be a pharmaceutically active agent (e.g., small molecule drug) and the heteronucleant may be an excipient (e.g., crystalline organic particles, polymer particles). In some such embodiments, the method may further comprise forming a pharmaceutical product (e.g., pharmaceutically acceptable product, pharmaceutically acceptable tablet) comprising the pharmaceutically active agent and the excipient. In some embodiments, the suspension may be fluidized as described above. In other instances, the control of transport properties and formation of free crystals that may serve a nucleants may be achieved using other mixing techniques. For example, a mixed-suspension, mixed-product-removal (MSMPR) crystallizer may be used. In certain embodiments, non-traditional propeller, such as a Visco Jet® agitator, that allow for a reduced velocity or reduced impact force compared to traditional blade propellers (e.g., stainless steel propeller).

Without being bound by theory, it is believed that selective heterogeneous crystallization is achieved using the methods and systems, described herein, due at least in part, to the ability to maintain relatively low supersaturation concentrations of the agent during crystallization and/or the utilization of relatively gentle mixing techniques. In general, the concentration of agent (e.g., pharmaceutically active agent) within the suspension may be maintained in a metastable zone of supersaturation. It is believed that such a relatively low supersaturation concentration inhibits primary nucleation that leads to bulk crystallization and promotes heteronucleation on a surface of the heteronucleant (e.g., excipient). For instance, in some embodiments, crystallizing a pharmaceutically active agent in a suspension comprising a solvent, an excipient, and the pharmaceutically active agent within in a metastable zone of supersaturation may result in the formation of a plurality of pharmaceutically active agent crystals, wherein greater than or equal to about 80 wt. % (e.g., greater than or equal to about 85 wt. %, greater than or equal to about 90 wt. %, greater than or equal to about 95 wt. %, or greater than or equal to about 99 wt. %) of the pharmaceutically active agent crystals are in contact with a surface of the excipient. Accordingly, fewer than or equal to about 20 wt. % (e.g., fewer than or equal to about 15 wt. %, fewer than or equal to about 10 wt. %, fewer than or equal to about 5 wt. %, or fewer than or equal to about 1 wt. %) of the crystal are formed via bulk crystallization. Those of ordinary skill in the art would be knowledgeable of techniques to determine the metastable zone of supersaturation for a wide variety of agents, including pharmaceutically active agents.

It is also believed that the utilization of relatively gentle mixing techniques contribute to the selectivity toward heteronucleation discovered to occur in certain of the methods and systems, described herein. In general, relatively gentle mixing techniques are believed to significantly reduce and/or eliminate secondary nucleation due to contact nucleation. As will be known to those of ordinary skill in the art, mixing of a suspension in a container will cause components of the suspension to contact solid surfaces within the container. The force at which one or more components contacts (e.g., impacts) a solid surface is dependent on numerous factors including the mixing method used. Under relatively harsh mixing conditions, contact nucleation may occur at least in part as a result of a force (e.g., frictional force, compressive force) being generated between a between one or more surfaces of the container and an agent crystal bound to heteronucleants. Without wishing to be bound by theory, it has been postulated that, in some instances, contact between an agent crystal and one or more surfaces results in removal of an adsorbed solute layer surrounding the agent crystal, leading to the generation of secondary nuclei and accordingly, secondary nucleation.

It has also been postulated that impact between one or more surfaces of the container and a component of the suspension, in some instances, may cause the component to fracture. For instance, in some embodiments, an agent crystal bound to heteronucleants may collide with one or more solid surfaces within the crystallizer. If the energy per volume absorbed by the agent crystal (e.g., pharmaceutically active agent crystals) as a result of a collision exceeds the mechanical strength and/or toughness of the agent crystal and/or exceeds the attachment energy (e.g., bond energy) of the agent crystal to the heteronucleant, the agent crystal will fracture. The fracture may lead to at least a portion of the agent crystal (e.g., the entire crystal) breaking away and forming a free crystal. The free crystal may serve as a nucleation surface and lead to secondary nucleation. It should be understood that the formation of free crystals due to fracture is distinct from the formation of isolated crystals, i.e., free crystal that usually do not serve as a heteronucleation surface, but may promote crystal growth.

In general, the impact force or energy per volume required to fracture a crystal is a material property of the crystal. One of skill in the art would be able to determine the maximum flow rate of the suspension to prevent the formation of a crystal fracture for a given agent crystal. In some embodiments, the velocity of the suspension may be controlled within a range that does not produce fracturing of a wide variety of crystalline materials. Accordingly, the velocity will result in an impact that does not fracture crystals.

In some embodiments, a method for selective heterogeneous crystallization of an agent on a heteronucleant may comprise agitating a suspension comprising an excipient and a dissolved pharmaceutically active agent in a crystallizer and while agitating, crystallizing the pharmaceutically active agent on at least a portion of a surface of the excipient to form pharmaceutically active agent crystals, such that during the agitation, fewer than or equal to about 20% of pharmaceutically active crystals collide with a solid surface within the crystallizer with a force of greater than 1.2 N. As another example, the velocity of the suspension may be selected such that selective heterogeneous crystallization may comprise agitating a suspension comprising an excipient and a dissolved pharmaceutically active agent in a crystallizer and while agitating, crystallizing the pharmaceutically active agent on at least a portion of a surface of the excipient to form pharmaceutically active agent crystals, such that during the agitation, fewer than or equal to about 20% of the pharmaceutically active crystals that collide with a solid surface fracture as a result of the collisions.

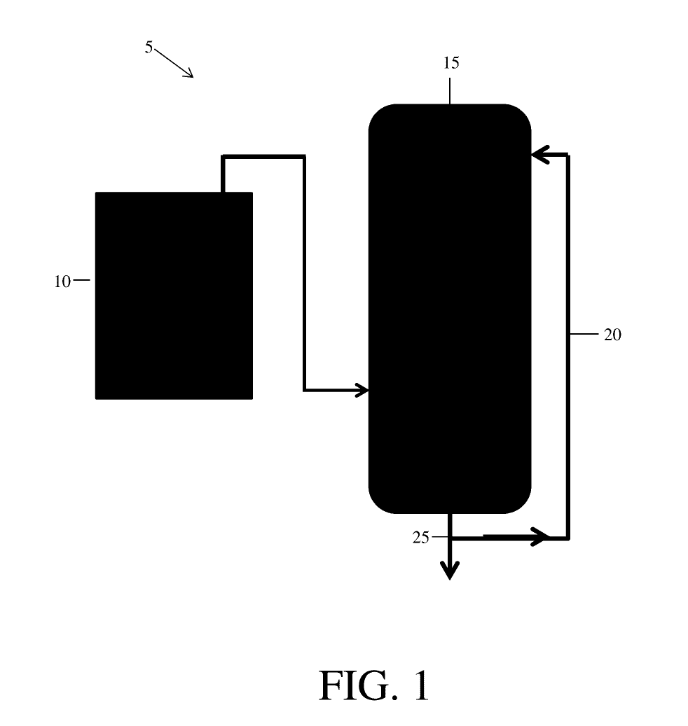

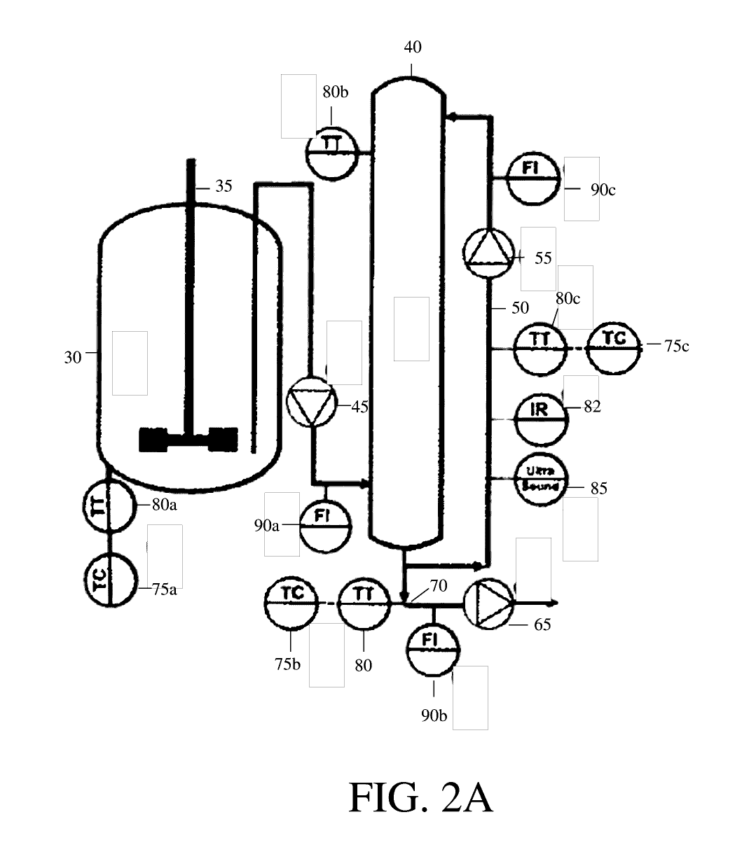

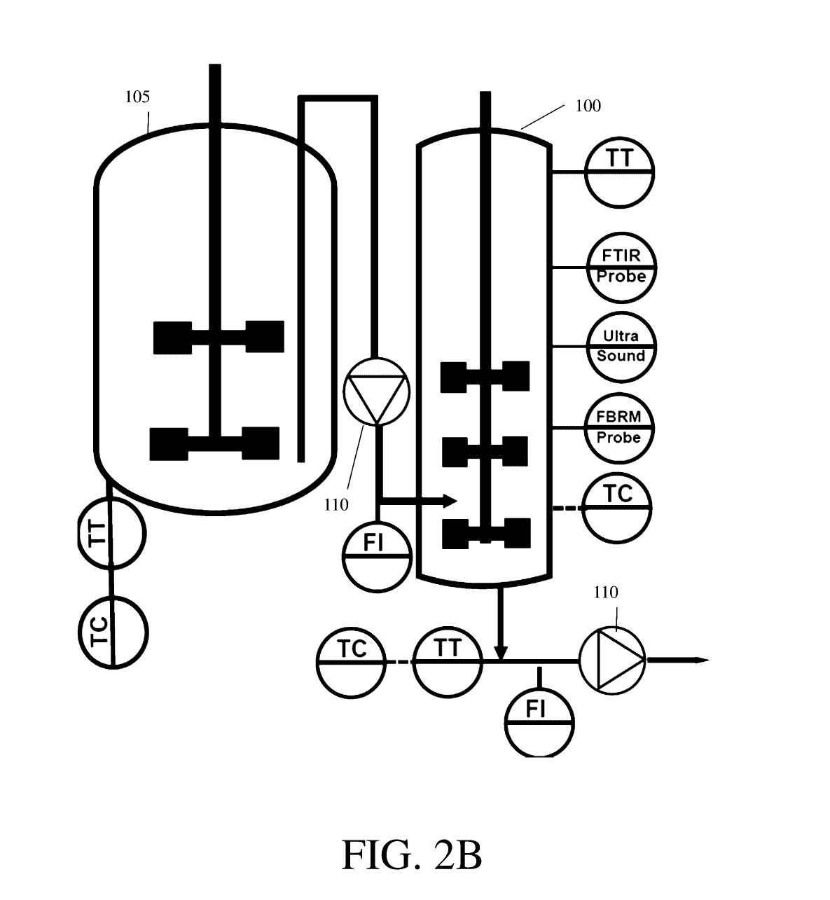

As mentioned above, in some embodiments, an agent may be crystallized on one or more surfaces of a heteronucleant in a crystallizer (e.g., continuous flow fluidized bed crystallizer). Non-limiting examples of crystallizers, described herein, are shown in FIGS. 1 and 2. Referring to some embodiments, a crystallizer 5 may comprise a feed tank 10 and a fluidization chamber 15 as shown in FIG. 1. In some embodiments, a slurry comprising an agent (e.g., pharmaceutically active agent) and heteronucleant (e.g., excipient) may be added to feed tank 10. The slurry from the feed tank may be flowed (e.g., via a pump, via a source of peristaltic pressure) into the fluidization chamber 15. In other embodiments, the slurry may be formed in the crystallizer instead of being added to the feed tank. In some such embodiments, the feed tank may contain and flow one or more component of the slurry (e.g., agent and solvent, heteronucleant) to the fluidization chamber. The remaining components of the slurry may be added to the fluidization chamber via other inlet means to form a slurry. Fluidization of the slurry may occur in the fluidization chamber to form a uniform or substantially uniform suspension of the slurry. The fluidized suspension may be circulated in the closed loop, 20, during crystallization for a desired time or to achieve a desired crystal size, as described above. Once the desired level of crystallization has occurred, at least a portion of the suspension or one or more components of the suspension (e.g., heteronucleant with bound agent crystals) may be removed via discharge line 25.

In some embodiments, the crystallizer may include additional optional features, such as sensors, as shown in FIG. 2A. As illustrated in FIG. 2A, a crystallizer may comprise feed tank 30 and an agitator 35 configured to maintain the uniformity of the slurry or slurry component in the feed tank. A pump (e.g., peristaltic pump) 45 may feed the slurry from the feed tank into the fluidization chamber 40. The fluidized suspension may be flowed within closed loop 50 using pump 55. In some embodiments, pump 55 circulates the suspension at a sufficient flow rate within a closed loop to maintain the uniformity of the suspension fluidized bed and/or allow for the requisite mass and heat transfer. After the desired crystallization period, the desired product (e.g., portion of the suspension, agent crystals bound to heteronucleant) may be removed from the fluidization chamber may be pumped out using pump 65 through the discharge line 70.

In some embodiments, the crystallizer may optionally comprise one or more temperature controllers (e.g., 75a, 75b, 75c) and temperature monitors (e.g., 80a, 80b, 80c). In some embodiments, feed tank 30, fluidization chamber 40, and the discharge line 70 are jacketed separately, and can be independently maintained at different temperatures. In some embodiments, the crystallizer may optionally comprise in-line concentration probes (e.g., 82 and 85). For instance, the crystallizer may optionally comprise an FTIR probe and/or an ultrasound probe. In some instances, the crystallizer may optionally comprise flow meters (e.g., 90a, 90b, and 90c).

In some embodiments, the operation of the crystallizer may be as follows. In one example, the feed tank is maintained at saturation or subsaturation temperature of the dissolved agent. The fluidization chamber and discharge lines may be maintained at a lower steady temperature to generate sufficient supersaturation. The feed may be fed to the column on a continuous basis, and the desired product may also be removed on a continuous basis at the same mass flow rate. The size of the fluidization chamber, throughput rate, inlet concentration, and column temperature may be tuned as desired. The desired product that exits the fluidization chamber may be filtered using a filter (e.g., continuous) to collect the crystals. In some embodiments, the filter may comprise a filtration medium configured to retain at least a portion of the particles (e.g., heteronucleant with bound agent crystals) above a threshold size and to pass particles (e.g., heteronucleant) and fluid below a threshold size. Filtration medium can have pores (e.g., straight-through pores, tortuous pores, mesh, etc.), and can be made of any suitable material, including materials suitable for use in pharmaceutical production. In certain embodiments, the filter may separate solids from liquids via porous plate and vacuum suction to remove one or more solvent. In some embodiments, agent crystals formed via bulk crystallization may be removed during filtration (e.g., based on their size). In some such cases, after removal via filtration, the agent crystals formed via bulk crystallization may be subjected to a process (e.g., heating) that allows these bulk agent crystals (e.g., to be dissolved recycled into suspension (e.g., via the feed tank) as dissolved agent (e.g., dissolved pharmaceutically active agent). In certain embodiments, agent crystals formed via bulk crystallization may be relatively small compared to agent crystals formed via heterogeneous crystallization.

In some embodiments, after filtration, the crystals may be dried and used in another process (e.g., tablet pressing). The drier may be configured to at least partially remove moisture and/or other liquids from the remaining solid. Drying may involve evaporation of liquid, e.g., via heating and/or the reduction of the pressure of the surrounding environment. Exemplary dryers include rotary drum dryers and screw dryers.

It should be understood that though the crystallizer has been described as using a fluidized bed, in some instances, other mixing techniques may be used. For example, a mixed-suspension, mixed-product-removal (MSMPR) crystallizer may be used as shown in FIG. 2B provided that relatively gentle mixing is utilized. The mixed-suspension, mixed-product-removal crystallizer may be arranged as described above with respect to the FIG. 2A, except the fluidization chamber may be replaced with a mixed-suspension, mixed-product-removal and a recirculation chamber may not be used. That, is the mixed-suspension, mixed-product-removal crystallizer may comprise a feed tank 105 and one or more pumps 110 to move one or more component in the feed tank to the mixed-suspension, mixed-product-removal chamber 100. The mixed-suspension, mixed-product-removal crystallizer may also comprise optional components, such as temperature controllers (e.g., TC), temperature monitors (TT), flow meters (F1), and/or various probes (e.g., ultrasound probe, FBMR probe, FTIR probe, In some instances, agents may be collected via filtration and drying as described above.

As another example, a non-traditional propeller that results in relatively gentle mixing may be used. In certain embodiments, a non-traditional propeller may comprise one or more members having an open structure instead of traditional blades. For instance, each traditional propeller blade may be replaced with members having an open structure. In one example, a non-traditional propeller (e.g., Visco Jet® agitator) may comprise one or more members (e.g., 3 members) that substantially have the shape of a truncated cone (i.e. cone having a frustum) instead of traditional blades. The shape of a truncated cone may be formed may a solid material (e.g., solid truncated cone) or a material having gaps (e.g., spiral in the shape of a truncated cone). In some embodiments, a non-traditional propeller may allow mixing and homogenization of suspensions using relatively low rotational speeds, and accordingly produce relatively small forces (e.g., shear forces, impact forces, and/or frictional forces). For instance, it is believed that a non-traditional propeller (e.g., Visco Jet® agitator) having truncated cone members (e.g., 3 members) instead of traditional blades has a venturi effect on the suspension being mixed such that suspension components in contact with the leading edge of the truncated cones are almost static. This effect is due in part to the generation of pressure waves in in front of, above and below the plane of rotation that produce strong circulating currents above and below the propeller. In some instances, agents may be collected via filtration and drying as described above. In other embodiments, non-fluidization mixing techniques may not be suitable.

In certain embodiments, the volume of the fluidization chamber can be selected as desired. For example, the fluidization chamber can have a volume of equal to or less than about 1,000 liters, equal to or less than about 750 liters, equal to or less than about 500 liters, equal to or less than about 250 liters, or equal to or less than about 100 liters equal to or less than about 75 liters, or equal to or less than about 50 liters, equal to or less than about 25 liters, or equal to or less than about 15 liters, equal to or less than about 10 liters, or equal to or less than about 5 liters, equal to or less than about 1 liter (and/or, in certain embodiments, equal to or greater than 10 milliliters, equal to or greater than 100 milliliters, or equal to or greater than 500 milliliter).

The fluidization chamber can, in some embodiments, be configured to contain (and/or, can contain during operation of the reactor) a volume of suspension of equal to or less than about 1,000 liters, equal to or less than about 750 liters, equal to or less than about 500 liters, equal to or less than about 250 liters, or equal to or less than about 100 liters equal to or less than about 75 liters, or equal to or less than about 50 liters, equal to or less than about 25 liters, or equal to or less than about 15 liters, equal to or less than about 10 liters, or equal to or less than about 5 liters, equal to or less than about 1 liter (and/or, in certain embodiments, equal to or greater than 10 milliliters, equal to or greater than 100 milliliters, or equal to or greater than 500 milliliter).

As described herein, in some embodiments, the methods and systems described herein may result in selective heterogeneous crystallization. For instance, in some embodiments, the percentage of crystals that form on at least a portion of a surface of the heteronucleant (e.g., excipient) may be greater than or equal to about 70 wt. %, greater than or equal to about 75 wt. %, greater than or equal to about 80 wt. %, greater than or equal to about 85 wt. %, greater than or equal to about 90 wt. %, greater than or equal to about 95 wt. %, greater than or equal to about 97 wt. %, greater than or equal to about 98 wt. %, or greater than or equal to about 99 wt. % of the percentage of crystals formed on the heteronucleant surface as may be determined by using high pressure liquid chromatography or by obtaining an optical and/or scanning electron microscopy image of a representative sample of the crystals and calculating the fraction of crystals on a heteronucleant surface. In some embodiments, the minimum weight percentage of crystals formed by heterogeneous crystallization is about 60 wt. %

In some embodiments, the percentage of crystals formed via bulk crystallization may be less than or equal to about 20 wt. %, less than or equal to about 18%, less than or equal to about 15 wt. %, less than or equal to about 12 wt. %, less than or equal to about 10 wt. %, less than or equal to about 8 wt. %, less than or equal to about 5 wt. %, less than or equal to about 3%, less than or equal to about 2 wt. %, less than or equal to about 1%, or less than or equal to about 0.5 wt. %. The percentage of crystals formed by bulk crystallization may be determined using FBRM. Crystals formed by bulk crystallization generally have a smaller size than crystals formed by heterogeneous crystallization. The agent crystals formed via bulk crystallization versus heterogeneous crystallization via nucleation on the heteronucleant can be quantified using a Focused Beam Reflectance Measurement (i.e., FBRM) probe, which measures the chord length distribution of crystals.

In some embodiments, the percentage of crystals formed via secondary nucleation (e.g., contact nucleation) may be less than or equal to about 20 wt. %, less than or equal to about 18 wt. %, less than or equal to about 15 wt. %, less than or equal to about 12 wt. %, less than or equal to about 10 wt. %, less than or equal to about 8 wt. %, less than or equal to about 5 wt. %, less than or equal to about 3 wt. %, less than or equal to about 2 wt. %, less than or equal to about 1%, or less than or equal to about 0.5 wt. %. The percentage of crystals formed by secondary nucleation may be determined using a Focused Beam Reflectance Measurement (i.e., FBRM) probe, as described above.

In general, the percentage of crystals formed via a bulk crystallization process may be determined as follows. First, FBRM is used to determine the particle size distribution of the heteronucleant fed into the fluidization chamber (e.g., heteronucleant prior to being used in a crystallization process). The particle distribution of the particles retained after crystallization process, as described herein, is also analyzed using a focused beam reflectance measurement probe. Crystallization of an agent on the surface of a heteronucleant increases the size of the heteronucleant particles. For example, in the case of complete heterogeneous nucleation, all the heteronucleant particles grow in size and the particle size distribution shift towards higher sizes. That is, the weight percentage of particles having the size of the heteronucleant itself disappears and the average size of all particles increases.

Typically, the particle size of heteronucleant is much larger than the size of bulk crystals. The presence of bulk crystals would lead to the appearance of small particles, which are often smaller than the smallest heteronucleant. From size analysis data, one can quantify the (1) volume fraction (V_{B}) of smallest crystals of agent (assumed to be bulk crystals) and (2) volume fraction of agent crystallized on the surface of heteronucleant (V_{H}). If V_{N} is the volume fraction of heteronucleant, then:

V_{N}+V_{H}+V_{B}=1;

the fraction of agent present as bulk crystals=V_{B}/(V_{H}+V_{B});

the fraction of agent crystallized on heteronucleant surface=V_{H}/(V_{H}+V_{B}); and

the mass fraction of agent in the sample=(V_{B}+V_{H})*rhoₐ/((V_{B}+V_{H})*rhoₐ+V_{N}*rhoₕ), where rhoₐ is the density of agent and rhoₕ is the density of heteronucleant.

In the above analysis, it is assumed that (1) all the bulk crystals are smaller than heteronucleant, and (2) the heteronucleant does not breaking into smaller crystals. To confirm this assumption, a known amount of agent crystals bound to one or more surfaces of a heteronucleant are dissolved in a solvent. The solvent dissolves only the agent. The solution of agent is analyzed for its concentration by using high-performance liquid chromatography (i.e., HPLC). This helps in finding the mass fraction of agent in the product. The mass fraction calculated using FBRM and HPLC are compared against each other. If the HPLC and FBRM data match within a certain error percentage (e.g., less than or equal to about 40%, less than or equal to about 30%, less than or equal to about 20%, less than or equal to about 10%, less than or equal to about 5%) the FBRM results are used to determine the weight percentages. If the HPLC and FBRM data do not match within the error percentage, image based analysis is carried out, where each particle on the image is analyzed for its identity (e.g., whether it is heteronucleant or agent) and its size. This data can be used in quantifying the fractions V_{N}, V_{H}, and V_{B}. Optical microscope, scanning electron microscope, particle vision measurement (PVM) may be used for obtaining images.

As noted above, in some embodiments, the velocity of the suspension may be selected to prevent fracture of the crystals. In some embodiments, a relatively low average velocity and/or speed may reduce the frequency of contact between an agent crystal bound to a heteronucleant and one or more surfaces of the container and/or the force of the contact. In some embodiments, the average velocity and/or speed of the suspension during crystallization and/or the average rotational speed applied to the suspension during crystallization may be less than or equal to about 60 mm/s, less than or equal to about 55 mm/s, less than or equal to about 50 mm/s, less than or equal to about 45 mm/s, less than or equal to about 40 mm/s, less than or equal to about 35 mm/s, less than or equal to about 30 mm/s, or less than or equal to about 25 mm/s. In some embodiments, the average velocity and/or speed of the suspension during crystallization and/or the average rotational speed applied to the suspension during crystallization may between about 10 mm/s and about 60 mm/s, between about 10 mm/s and about 55 mm/s, between about 10 mm/s and about 50 mm/s, between about 10 mm/s and about 40 mm/s, between about 10 mm/s and about 30 mm/s, between about 12 mm/s and about 30 mm/s, between about 14 mm/s and about 30 mm/s, between about 15 mm/s and about 30 mm/s, between about 16 mm/s and about 30 mm/s, between about 18 mm/s and about 30 mm/s, or between about 20 mm/s and about 30 mm/s. The average velocity may be determined by using a tracer and measuring the residence time distribution of the tracer. It should be understood that the average velocity may, in some instances, depend on the configuration of the crystallizers and other suitable values of average velocity and/or speed may be possible.

In some embodiments, the average impact force between an agent crystal and one or more solid surfaces within the crystallizer during crystallization may be less than or equal to about 1.2 N (e.g., less than or equal to about 1.0 N, less than or equal to about 0.9 N, less than or equal to about 0.8 N, less than or equal to about 0.7 N, less than or equal to about 0.6 N). In some embodiments, the impact force may be between about 0.5 N and about 1.2N.

In some embodiments, crystallization methods, described herein may result in a relatively high drug loading. As used herein, “drug loading” refers to the weight of the agent per unit weight of the product. In some embodiments, the drug loading may be greater than or equal to about 5 wt. %, greater than or equal to about 10 wt. %, greater than or equal to about 20 wt. %, greater than or equal to about 30 wt. %, greater than or equal to about 40 wt. %, greater than or equal to about 50 wt. %, greater than or equal to about 60 wt. %, greater than or equal to about 70 wt. %, greater than or equal to about 80 wt. %, or greater than or equal to about 85 wt. %. In some embodiments, the drug loading may be between about 5 wt. % and about 90 wt. %, between about 10 wt. % and about 90 wt. %, between about 20 wt. % and about 90 wt. %, between about 30 wt. % and about 90 wt. %, between about 40 wt. % and about 90 wt. %, or between about 50 wt. % and about 90 wt. %.

In some embodiments, the heteronucleant (e.g., excipient) may be chosen based on its ability to promote crystallization, preferentially nucleate a specific polymorph, and/or its solubility properties. In some instances, the heteronucleant may also be selected to be a pharmaceutically acceptable excipient. In general, any suitable heteronucleants may be used. Suitable heteronucleants are described in the following, which are herein incorporated by reference in their entirety for all purposes: U.S. Patent Application Publication No. US2012/0076860, filed Aug. 23, 2011, and entitled “Compositions, Methods, and Systems Relating to Controlled Crystallization and/or Nucleation of Molecular Species,” and U.S. Patent Application Serial No. US 2013/0118399, filed Nov. 15, 2012, and entitled “Methods and Systems Relating to the Selection of Substrates Comprising Crystalline Templates for the Controlled Crystallization of Molecular Species.”

In some embodiments, the heteronucleant may be a biologically compatible material, or another material that can be used as an excipient for a pharmaceutically active species. In certain embodiments, the heteronucleant may be porous. The porous material may be any material that contains various pores within which a pharmaceutically active agent may be formed. In some cases, a non-porous material may be processed to include a plurality of pores. In other embodiments, the heteronucleant may not be porous.

In some embodiments, the heteronucleant may be, for example, a polymeric material. In some cases, the heteronucleant may comprise an organic material. In some cases, the heteronucleant may consist of an organic material (e.g., sugar alcohol). In some cases, the heteronucleant may consist essentially of an organic material. For example, the heteronucleant may be a crystalline sugar alcohol. In some cases, the heteronucleant may comprise an inorganic material. In some cases, the heteronucleant may consist of an inorganic material. In some cases, the heteronucleant may consist essentially of an inorganic material. The heteronucleant may include materials which are substantially soluble in aqueous solutions.

Examples of heteronucleants, include, but are not limited to, starches (e.g., corn starch, potato starch, pre-gelatinized starch, or others), gelatin, natural and synthetic gums (e.g., acacia, sodium alginate, alginic acid, other alginates, powdered tragacanth, guar gum), lactose including hydrates thereof (e.g., lactose monohydrate), mannitol, dextrin, dextrates, cellulose and its derivatives (e.g., ethyl cellulose, hydroxyethyl cellulose, cellulose acetate, carboxymethyl cellulose calcium, sodium carboxymethyl cellulose, methyl cellulose, hydroxypropyl methyl cellulose, microcrystalline cellulose), polyvinyl pyrrolidone (or povidone), polyethylene oxide, polydextrose, polyoxamer, metal carbonates (e.g., magnesium carbonate) metal oxides (e.g., silicon dioxide, titanium dioxide, aluminum oxide, etc.), clays (e.g., bentonite, talc), other sugars, certain salts, other glass materials, mixtures thereof, and the like. In some cases, the heteronucleant comprises cellulose, cellulose acetate, carbon, silicon dioxide, titanium dioxide, aluminum oxide, clays, other glass materials, or combinations thereof. In one set of embodiments, the heteronucleant comprises cellulose. In one set of embodiments, the heteronucleant comprises silicon dioxide. In some embodiments, the substrate comprises a heteronucleant. The heteronucleant may form a hydrogel. In some cases, the polymeric material is porous. The polymeric material may also be formed such that at least one surface of the polymeric material comprises surface features to aid in the crystallization and/or nucleation processes.

In some embodiments, heteronucleant may be any material that has the desired cell parameter, desired space group, desired functional groups, and is substantially insoluble in the solvent(s) in the suspension, as desired in more detail below.

In some embodiments, the heteronucleant comprises a material, wherein the material comprises a crystalline template. The term “crystalline” or “crystal” as used herein is given its ordinary meaning in the art and refers to a material which exhibits uniformly arranged molecules or atoms. Methods of determining whether a material is crystalline are known in the art, for example, x-ray diffraction techniques. A heteronucleant comprising a crystalline template refers to a substrate in which at least one surface is crystalline. In some embodiments, the crystalline template is selected so as to 1) have a complimentary space group as compared to the space group of the polymorph to be crystallized, 2) have complimentary unit cell dimensions as compared to the polymorph to be crystallized, and/or 3) comprise a plurality of complimentary functional groups on at least one surface of the substrate, wherein the functional groups are complimentary to functional group(s) of the molecular species.

In some embodiments, the space group of the crystalline template is complimentary to the space group of the polymorphic form of the molecular species to be crystallized. In some embodiments, the space group of the crystalline template is the same as the space group of the polymorphic form of the molecular species to be crystallized. The term “space group” is given its ordinary meaning in the art and refers to a group or array of operations consistent with an infinitely extended regularly repeating pattern. Generally, the space group is the symmetry of a three-dimensional structure, or the arrangement of symmetry elements of a crystal. There are approximately 230 known space groups.

In some embodiments, the unit cell dimensions of the crystalline template of the heteronucleant are complimentary to the unit cell dimensions of the polymorphic form of the molecular species to be crystallized. The term “unit cell” is given its ordinary meaning in the art and refers to the portion of a crystal structure that is repeated infinitely by translation in three dimensions. Generally, a unit dimensions is characterized by three vectors (e.g., A, B, and C as used herein for the unit cell dimensions of the polymorph of the molecular species, or X, Y, and Z as used herein for the unit cell dimensions of the crystalline template), wherein the three vectors are not located in one plane and form the edges of a parallelepiped. Angles alpha, beta, and gamma define the angles between the vectors: angle alpha is the angle between vectors B and C or Y and Z, angle beta is the angle between vectors A and C or X and Z, and angle gamma is the angle between vectors A and B or X and Y. The entire volume of a crystal can be constructed by regular assembly of unit cells; each unit cell comprises a complete representation of the unit of pattern, the repetition of which builds up the crystal.

In some embodiments, the complimentary unit cell dimensions of the crystalline template are selected as follows. In the following description, the unit cell dimensions of the crystalline template has vectors X×Y×Z and the unit cell dimensions of the polymorph of the molecular species to be crystallized has vectors A×B×C. In some embodiments, vectors X, Y, and Z are selected so as to have a dimensions which are equal to or close to the dimensions of vectors A, B, and C, respectively. In some embodiments, the crystalline substrate is selected so that X=A±(R×S); Y=B±(R×S); and Z=C±(R×S), wherein S is a tolerance factor and R is the longest of A, B, and C. In some embodiments, S is between 0 and 0.1, or between 0 and 0.09, or between 0 and 0.08, or between 0 and 0.07, or between 0 and 0.06, or between 0 and 0.05, or between 0 and 0.04, or between 0 and 0.03, or between 0 and 0.02, or between 0 and 0.01. In one embodiment, S is between 0 and 0.05. In another embodiment, S is between 0 and 0.03.

In some embodiments, the heteronucleant material may be selected such that at least one surface (e.g., comprising the crystalline template) of the heteronucleant comprises a plurality of at least one type of functional group which is complimentary to at least one functional group of the small organic molecule. That is, the functional groups of the heteronucleant may be selected so as interact with a specific functional group of the organic small molecule of interest. Complimentary types functional groups (e.g., comprised on the surface of the heteronucleant and the molecular species) will be known to those of ordinary skill in the art. The association may be based on formation of a bond, such as an ionic bond, a covalent bond (e.g., carbon-carbon, carbon-oxygen, oxygen-silicon, sulfur-sulfur, phosphorus-nitrogen, carbon-nitrogen, metal-oxygen, or other covalent bonds), a hydrogen bond (e.g., between hydroxyl, amine, carboxyl, thiol, and/or similar functional groups), a dative bond (e.g., complexation or chelation between metal ions and monodentate or multidentate ligands), Van der Waals interactions, or the like.

In some cases, the heteronucleant material may be selected such that it comprises at least a plurality of hydroxyl functional groups, a plurality of carboxylic acid ester functional groups, a plurality of nitrogen containing base functional group, a plurality of aryl (e.g., phenyl) functional groups, a plurality of carboxyl functional group, a plurality of tertiary amide functional groups, or combinations thereof. As a non-limiting example, if the small organic molecule comprises an aryl group, the functional group on the surface of the heteronucleant may be selected to be an aryl functional group, such that pi-interactions can occur between the surface of the heteronucleant and the small organic molecule. As another example, if the small organic molecule comprises a hydrogen-bond donating group, the functional group on the surface of the heteronucleant may be selected to be a hydrogen-bond accepting group. As a specific example, the small organic molecule may contain a carboxylic acid functionality and the surface of the heteronucleant may contain a tertiary amide functionality. As another specific example, the small organic molecule may contain a carbonyl group and the surface of the heteronucleant may contain a hydroxyl group. As yet another specific example, both the small organic molecule and the surface of the heteronucleant may contain phenyl groups, and the interaction may be a pi-stacking interaction.

In some embodiments, in addition to selecting a heteronucleant based on the surface chemistry, the morphology of the heteronucleant can also be varied to affect the crystallization and/or nucleation of a molecular species (e.g., small organic molecule). The morphology of a heteronucleant may be varied by changing 1) the outer surface morphology (e.g., features such as wells) and/or 2) the inner surface morphology (e.g., such that the crystallization heteronucleant is porous). In some embodiments, the outer surface morphology of the heteronucleant may be selected so as to promote crystallization (e.g., by increasing the induction rate and/or by promoting the formation of a certain crystal form) of a selected crystal form of an agent (e.g., small organic molecule). At least one outer surface of the heteronucleant may comprise a plurality of features having a shape which is complimentary to a known crystal form (e.g., polymorph) of the small organic molecule. For example, if a crystal form is known for a small organic molecule, the shape and/or angle(s) of the crystals are known or can be deduced/calculated. Based at least in part on the knowledge of the shape and/or angle(s) of the crystals, a complimentary shape and/or angle(s) of a plurality of features formed in the surface of the crystallization heteronucleant may be selected.

Suitable substrates for use in the methods and systems as described herein are known in the art. In some embodiments, the substrate may comprise a crystalline material. In some embodiments, the substrate comprises a material which is found in the Cambridge Structural Database. In some cases, the substrate is not soluble in the solution in which the crystallization is to occur.

In general, the heteronucleant is insoluble in the solvent in which the agent (e.g., pharmaceutically active agent) is dissolved within and is used to form the suspension. For instance, in some embodiments, the solubility of the heteronucleant in the solvent is less than about 1 mg/L, less than about 0.75 mg/L, less than about 0.5 mg/L, less than about 0.25 mg/L, less than about 0.1 mg/L, less than about 0.05 mg/L, less than about 0.01 mg/L, less than about 0.005 mg/L, or less than about 0.001 mg/L.

In some embodiments of the present invention, the suspension composition, including the solvent, the heteronucleant, and the agent (e.g., pharmaceutically active agent), may be selected such that the agent has a stronger interaction/affinity with the heteronucleant as compared to the solvent. The rate of crystallization and/or nucleation may be increased in embodiments where the agent has preferred interactions with the heteronucleant over the solvent, as compared to embodiments where there is no preferential interactions. In addition, the interaction/affinity between the agent and the solvent may be greater than the interaction/affinity between the solvent and the heteronucleant. Without wishing to be bound by theory, a greater interaction of the agent with the heteronucleant as compared to any of the other interactions in the system (e.g., between the agent (e.g., small organic molecule) and the solvent, between the heteronucleant and the solvent) may aid in reducing the average induction time, as the agent is drawn towards the heteronucleant, and hence increases the chances of nucleation. Those of ordinary skill in the art will be capable of selecting combinations of solvents and heteronucleant materials for a selected agent (e.g., small organic molecule), based on the teaching described herein, which have the desired affinities/interactions between the solvent, the agent, and the heteronucleant.

The heteronucleant may be of any suitable shape, size, or form. In some cases, the heteronucleant may be a planar surface and/or a portion of a container. Non-limiting examples of shapes include sheets, cubes, cylinders, hollow tubes, spheres, and the like. In some cases, the maximum dimension of the heteronucleant in one dimension may be at least about 1 mm, at least about 1 cm, at least about 5 cm, at least about 10 cm, at least about 1 m, at least about 2 m, or greater. In some cases, the minimum dimension of the heteronucleant in one dimension may be less than about 50 cm, less than about 10 cm, less than about 5 cm, less than about 1 cm, less than about 10 mm, less than about 1 mm, less than about 1 um, less than about 100 nm, less than about 10 nm, less than about 1 nm, or less.

In some cases, the heteronucleant (e.g., excipient) may comprise a plurality of particles (e.g., polymeric particles). In some cases, a particle may be a nanoparticle, i.e., the particle has a characteristic dimension of less than about 1 micrometer, where the characteristic dimension of a particle is the diameter of a perfect sphere having the same volume as the particle. The plurality of particles, in some embodiments, may be characterized by an average diameter (e.g., the average diameter for the plurality of particles). In some embodiments, the diameter of the particles may have a Gaussian-type distribution. In some cases, the plurality of particles may have an average diameter of less than about an average diameter of less than about 5 mm, or less than about 4 mm, or less than about 3 mm, or less than about 2 mm, or less than about 1 mm, or less than about 500 um, or less than about 100 um, or less than about 50 um, or less than about 10 um, or less than about 1 um, or less than about 800 nm, or less than about 500 nm, or less than about 300 nm, or less than about 250 nm, or less than about 200 nm, or less than about 150 nm, or less than about 100 nm, or less than about 50 nm, or less than about 30 nm, or less than about 10 nm, or less than about 3 nm, or less than about 1 nm, in some cases. In some embodiments, the particles may have an average diameter of at least about 5 nm, at least about 10 nm, at least about 30 nm, at least about 50 nm, at least about 100 nm, at least about 200 nm, at least about 500 nm, at least about 800 nm, at least about 1000 nm, at least about 10 um, at least about 50 um, at least about 100 um, at least about 500 um, at least about 1 mm, at least about 2 mm, at least about 3 mm, at least about 4 mm, at least about 5 mm, or greater. In some cases, the plurality of the particles have an average diameter of about 10 nm, about 25 nm, about 50 nm, about 100 nm, about 150 nm, about 200 nm, about 250 nm, about 300 nm, about 500 nm, about 800 nm, about 1 mm, about 2 mm, about 3 mm, about 4 mm, about 5 mm, or greater.

In some embodiments, the average diameter of the crystals formed using the methods and systems, described herein, is less than or equal to about 100 microns, less than or equal to about 10 microns, less than or equal to about 1 micron, less than or equal to about 0.8 microns, less than or equal to about 0.6 microns, less than or equal to about 0.4 microns, less than or equal to about 0.2 microns, less than or equal to about 0.1 microns, less than or equal to about 0.08 microns less than or equal to about 0.05 microns, or less than or equal to about 0.02 microns. In some instances, the average diameter of the crystals may be between about 0.01 microns and about 100 microns, between about 0.01 microns and about 10 microns, between about 0.01 microns and about 1 micron, or between about 0.01 microns and about 0.4 microns. In some embodiments, the minimal average crystal diameter may be 0.1 microns.

In some embodiments, the coefficient of variation in the average crystal diameter is less than or equal to about 40%, less than or equal to about 35%, less than or equal to about 30% or less than or equal to about 25% less than or equal to about 20%, less than or equal to about 15%, less than or equal to about 10% or less than or equal to about 5%.

Crystallization of agents (e.g., pharmaceutically active agents, small organic molecules) may be carried out according to methods known to those of ordinary skill in the art. In some cases, a heteronucleant (e.g., as described herein) may be exposed to a solution comprising an agent (e.g., small organic molecule). Generally, the agent (e.g., pharmaceutically active agent) is substantially soluble in the solvent selected. In some cases, the solution comprising the solvent and the small organic molecule may be filtered prior to exposing the solution to the heteronucleant. The small organic molecule may be present in the solvent at a concentration of about 0.05 M, about 0.1 M, about 0.2 M, about 0.3 M, about 0.4 M, about 0.5 M, about 0.75 M, about 1 M, about 2 M, or greater. Non-limiting examples of solvents include water, acetone, ethanol, acetonitrile, benzene, p-cresol, toluene, xylene, mesitylene, diethyl ether, glycol, petroleum ether, hexane, cyclohexane, pentane, dichloromethane (methylene chloride), chloroform, carbon tetrachloride, dioxane, tetrahydrofuran (THF), dimethyl sulfoxide, dimethylformamide, hexamethyl-phosphoric triamide, ethyl acetate, pyridine, triethylamine, picoline, and combinations thereof.

Those of ordinary skill in the art will be aware of methods for inducing crystallization. For examples, in some cases, a system comprising a substrate and a solution comprising the small organic molecule may be cooled. Alternatively, the solution comprising the small organic molecule may be concentrated (e.g., by evaporation of at least a portion of the solvent). In another set of embodiments, a material that facilitates growth of a crystal (e.g., a non-solvent, anti-solvents, surfactants) may be added to the solution.

The methods and/or compositions of the present invention may find application relating to pharmaceutical compositions and/or methods, wherein the agent is a pharmaceutically active agent. As will be known to those of ordinary skill in the art, uniformity of the crystal size of the pharmaceutically active agents significantly affect a variety of different properties including solubility, bioavailability, and/or stability Accordingly, the ability to control the uniformity of the pharmaceutically active agent crystals (e.g., using the methods and systems described herein) provides the advantage of having the capability to form a relatively uniform final product. For embodiments where the crystals of the pharmaceutically active agent are not to be separated from the heteronucleant, the heteronucleant may be substantially non-toxic and/or bioabsorbable.

The term “bulk crystallization” has its ordinary meaning in the art and may refer to crystallization that results from a nucleation process that does not occur on a heteronucleant surface.

The term “excipient” as used herein refers to an inactive substance that serves as the vehicle or medium for a pharmaceutically active agent or other active substance.

The term “fracture” as used herein has its ordinary meaning in the art and may refer to the separation of a material into two or more pieces or the formation of a discontinuity, such as a split or crack in the material.

The term “small molecule” is art-recognized and refers to a composition which has a molecular weight of less than about 2000 g/mole, or less than about 1000 g/mole, and even less than about 500 g/mole. Small molecules may include, for example, nucleic acids, peptides, polypeptides, peptide nucleic acids, peptidomimetics, carbohydrates, lipids or other organic (carbon containing) or inorganic molecules. Many pharmaceutical companies have extensive libraries of chemical and/or biological mixtures, often fungal, bacterial, or algal extracts, which can be screened with any of the assays of the invention. The term “small organic molecule” refers to a small molecule that is often identified as being an organic or medicinal compound, and does not include molecules that are exclusively nucleic acids, peptides, or polypeptides. In some cases, the small organic molecule is a pharmaceutically active agent (i.e., a drug). A pharmaceutically active agent may be any bioactive agent. In some embodiments, the pharmaceutically active agent may be selected from “Approved Drug Products with Therapeutic Equivalence and Evaluations,” published by the United States Food and Drug Administration (F.D.A.) (the “Orange Book”). In a particular embodiment, the pharmaceutically active agent is aspirin or acetaminophen.

The compositions and/or crystals described herein may be used in “pharmaceutical compositions” or “pharmaceutically acceptable” compositions, which comprise a therapeutically effective amount of one or more of the polymers or particles described herein, formulated together with one or more pharmaceutically acceptable carriers, additives, and/or diluents. The pharmaceutical compositions described herein may be useful for diagnosing, preventing, treating or managing a disease or bodily condition including conditions characterized by oxidative stress or otherwise benefitting from administration of an antioxidant. Non-limiting examples of diseases or conditions characterized by oxidative stress or otherwise benefitting from administration of an antioxidant include cancer, cardiovascular disease, diabetes, arthritis, wound healing, chronic inflammation, and neurodegenerative diseases such as Alzheimer Disease.

The phrase “pharmaceutically acceptable” is employed herein to refer to those structures, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid, gel or solid filler, diluent, excipient, or solvent encapsulating material, involved in carrying or transporting the subject compound, e.g., from a device or from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

As used herein, the term “pharmaceutically active agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Pharmaceutically active agents include, without limitation, agents listed in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005. Preferably, though not necessarily, the pharmaceutically active agent is one that has already been deemed safe and effective for use in humans or animals by the appropriate governmental agency or regulatory body. For example, drugs approved for human use are listed by the FDA under 21 C.F.R. §§330.5, 331 through 361, and 440 through 460, incorporated herein by reference; drugs for veterinary use are listed by the FDA under 21 C.F.R. §§500 through 589, incorporated herein by reference. All listed drugs are considered acceptable for use in accordance with the present invention. In certain embodiments, the pharmaceutically active agent is a small molecule. Exemplary pharmaceutically active agents include, but are not limited to, anti-cancer agents, antibiotics, anti-viral agents, anesthetics, anti-coagulants, inhibitors of an enzyme, steroidal agents, steroidal or non-steroidal anti-inflammatory agents, antihistamine, immunosuppressant agents, antigens, vaccines, antibodies, decongestant, sedatives, opioids, pain-relieving agents, analgesics, anti-pyretics, hormones, prostaglandins, etc.

As used herein, a “subject” or a “patient” refers to any mammal (e.g., a human), for example, a mammal that may be susceptible to a disease or bodily condition. Examples of subjects or patients include a human, a non-human primate, a cow, a horse, a pig, a sheep, a goat, a dog, a cat or a rodent such as a mouse, a rat, a hamster, or a guinea pig. Generally, the invention is directed toward use with humans. A subject may be a subject diagnosed with a certain disease or bodily condition or otherwise known to have a disease or bodily condition. In some embodiments, a subject may be diagnosed as, or known to be, at risk of developing a disease or bodily condition.

The following examples are intended to illustrate certain embodiments of the present invention, but do not exemplify the full scope of the invention.

### EXAMPLES

### Example 1

Active Pharmaceutical Ingredients (API) are biologically active molecules present in any final drug product (tablets, capsules, syrups, etc.). Tablets, for example, typically contain API mixed with a solid excipient and other additives as needed to achieve the desired size, strength, friability, dissolution rate, etc. Ensuring the presence of the appropriate physical form (polymorph) of a given API, its purity, and dosage amount are crucial in any drug formulation.

Crystallization is the easiest way of producing molecules in their purest form. Therefore it is one of the most important unit operations in a majority of API manufacturing processes. In a typical tablet manufacturing facility, API molecules are crystallized in a batch crystallizer. The product slurry is filtered to separate out the crystals from the mother liquor in which the impurities ideally remain dissolved in solution. The crystals often undergo many of the following potential processing steps including but not limited to: drying, wet and dry granulation, roller compaction, milling, sieving, blending with excipient and other additives, etc., and ultimately powder compaction into the tablets. Regulatory agencies continue to desire and eventually require better control over processes and drug products with tighter specifications (e.g. ±5% of the label claimed dose [ICH guideline]).

Batch based operation and solid state blending, both can lead to an inhomogeneous final formulation. As an example, if one aims to produce a 250 mg tablet containing 50±5 mg drug, one might end up producing at least few tablets that contain less than 45 mg, and/or containing more than 55 mg of drug in them. It is not feasible to characterize each tablet for its composition. Hence, ensuring tight control over product quality is an important and challenging task.

The main goals of the crystallization process are to produce the material in the desired crystal form at high yield with a suitable crystal size distribution that allows for easy and efficient filtration as well as good blending with excipients to achieve content uniformity in the final drug product. Often meeting both of these goals is not possible as the crystals are difficult to filter because their size is too small or they have a needle shape. If the crystals are made large to aid in filtering they might not be suitable for blending without a milling process.

In this example, develop a process which allows crystallization of an API directly on a crystalline excipient surface. This aids in filtration, and eliminates the need for blending. As a result of the API being grown directly on the excipient surface the content uniformity obtained is better than in traditional processing methods. The composite particles produced are sent to a direct compression process where they are formed into tablets.

A Fluidized bed crystallizer (FBC) was utilized in the present invention, to carry out preferential heterogeneous crystallization of an API on an excipient surface, in a continuous manner. Continuous operation and heterogeneous crystallization help in robust control of the composition of the final drug formulation. Heterogeneous nucleation also decreases the probability of having undesired polymorphs. Fluidized Bed Crystallizers (FBC) are traditionally used as ‘growth only crystallizers’, where crystal seeds grow in a supersaturated solution. This example describes a new way of using FBC for a totally new concept of ‘heterogeneous nucleation only crystallizer’.

The API-Excipient-Solvent selection was important to this process. The specific API chosen was soluble in the solvent; however the excipient was insoluble in the same solvent. The API-Excipient combination was such that the API molecules easily nucleate on the surface of the excipient. Use of low supersaturation stopped primary nucleation, and ensures the presence of heterogeneous nuclei alone. These nuclei grew further during their stay inside the column. Tuning of residence time and supersaturation helps in tuning of the loading (weight of API per unit weight of product) of excipient particles with drug.

A schematic of the Continuous Fluidized Bed Crystallizer (CFBC) was shown in FIG. 2A. The CFBC contains a feed tank (30) and the fluidized column (40) as the two main components. The feed tank contained a solution of a given API dissolved in solvent mixed with a solid excipient. The excipient did not dissolve in the solvent used, and hence forms a slurry. To ensure uniform composition of the feed slurry, the feed tank was mounted with an agitator, and was continuously agitated. Pump 40 fed the slurry from the feed tank into the fluidized column (or crystallizer). Pump 55 circulated the slurry at a sufficient flow rate within a closed loop to ensure (1) uniform suspension of slurry in the fluidized bed, and (2) efficient mass transfer. The product from the fluidized crystallizer was pumped out using pump 65. The feed tank, fluidized column, and the discharge line were jacketed separately, and could be maintained at different temperatures, independently. Temperature monitors (80a, 80b, and 80c), and temperature controllers (75a, 75b, and 75c), in-line concentration probes (82 and 85) and flow meters (90a, 90b and 90c) helped in online monitoring and control of the entire crystallizer.

The feed tank was maintained at saturation or subsaturation temperature of the API solution. The fluidized column and discharge lines were maintained at a lower steady temperature to generate sufficient supersaturation. The excipient was chosen such that it promoted nucleation of API on its surface without causing any primary nucleation. The feed was fed to the column on a continuous basis, and the product was also removed on a continuous basis at the same mass flow rate. The size of the fluidized column, throughput rate, inlet concentration, and column temperature were tuned to match the dosage requirements. The product slurry was filtered using a continuous filter. The crystals were dried and sent to a tablet pressing unit.

The continuous and steady state operation, online monitoring of the process parameters, and their precise control using process analytical technology (PAT), made the manufacturing process and product quality easier to control and could be designed to result in higher quality with less effort than typically needed for batch processes. Absence of an impeller minimized secondary nucleation. Operation of the crystallizer in the metastable zone of supersaturation (low supersaturation where nucleation was absent) ensured the absence of primary nucleation. Heterogeneous nucleation on the added excipient surface ensured a more uniform composition of API throughout the material produced, which would yield a drug product with better content uniformity. Careful selection of excipients helped in crystallization of preferred polymorph on the surface of excipient, thus minimizing the uncertainties related to crystallizing a wrong polymorph.

In this, continuous manufacturing of API directly coated on excipient surface, increasing product uniformity, was successfully demonstrated. X-ray powder diffraction (XPRD), Differential scanning calorimetry (DSC), image analysis, high-performance liquid chromatography (HPLC), and density analysis were employed to determine the structure of the crystallized API, and its dosage composition.

Thus, with a minimum processes operations, a highly uniform drug product was achieved. This approach has multiple advantages over the conventional ways of producing API. In a conventional batch based system, dried API crystals are often milled to reduce their size, and later mixed with an excipient to meet the dosage requirements. In this example, direct deposition of API on an excipient surface achieved uniform composition of drug in the final formulation with a minimum of unit operations. Herein a direct physical bonding of API with the excipient is described. In conventional techniques, partial physical bonding might happen after blending, and tableting. However, it might not be as good and as uniform as it was in this example.

Preferential and energetically favorable heterogeneous nucleation of API on the surface of excipient can also ensure crystallization of selective polymorphs, thus ensuring presence of the required polymorph in the final formulation. In traditional approaches, making the formulation with the desired polymorph might be challenging, especially when the desired polymorph was not the most stable form.

Direct crystallization of an API on an excipient surface avoids time and energy intensive solid processing steps (milling, blending, and sieving of API crystals followed by their granulation), thus making the process much more economical. Direct nucleation also ensures minimal coagulation of particles, and hence, higher effective surface area of the drug and potentially better bio-availability.

This example encompassed two parts, the process itself, and the product produced. In terms of the process, a novel crystallization process with a new concept for a continuous fluidized bed crystallizer was developed. In terms of the product, the API was directly crystallized on the excipient surface, and therefore had different properties than traditionally prepared tablets.

This example not only makes the crystallization continuous, but also avoids the energy intensive unit operations such as milling, blending, sieving, and granulation. Continuous operation also ensures smaller equipment, and lower operating and maintenance costs.

Careful selection of API and excipient combination, right operating conditions, and continuous operation of the crystallizer with the application of online process analytical technology, helped in achieving the consistent product quality in the pharmaceutical industry.

An exemplary embodiment was implemented with Acetaminophen-D-mannitol-Ethanol as a sample API-Excipient-Solvent system. Acetaminophen was soluble in ethanol; however D-mannitol was insoluble in ethanol. D-mannitol had a crystal structure that was favorable for heterogeneous nucleation of acetaminophen.

### Materials and Methods

Feed Slurry Preparation:

D-mannitol (Sigma-Aldrich) as obtained directly from the chemical suppliers did not have smooth surfaces that were necessary to promote heterogeneous nucleation. Hence, we carried out temperature cycling of the raw D-mannitol, to improve the quality of its surfaces. In a typical temperature cycling experiment D-mannitol was dissolved and recrystallized in ethanol (200 proof KOPTEC)-deionized water mixture in cycles, by increasing and decreasing the temperature in cycles. This leads to dissolution of smaller particles, and subsequent growth of bigger particles. The result being a narrowing of size distribution of crystals and crystals with surfaces which promote the nucleation of acetaminophen on them. The narrow size distribution of crystals also helps in better and easier filtration of product. A solution of acetaminophen (Sigma-Aldrich) in ethanol was prepared by dissolving 196 mg of acetaminophen (AAP) per 1 g of ethanol.

Start-Up and Continuous Operation of FBC:

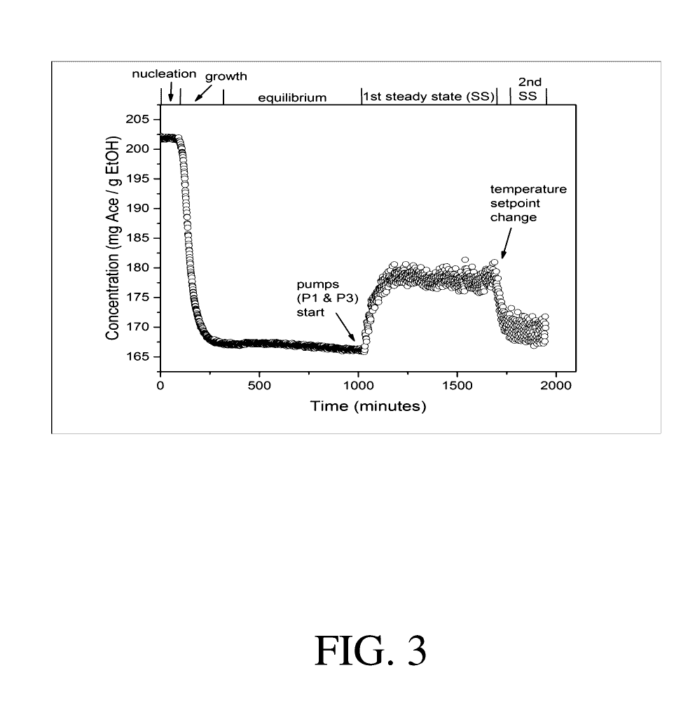

The API solution and D-mannitol were added to the jacked feed tank maintained at 25° C. (for every 100 g of API solution, 8 g of temperature cycled D-mannitol crystals were added). 573 ml of feed was added to the crystallizer maintained at 25° C. As the feed was added, the recirculation pump was also started to fluidize the slurry. The temperature of the crystallizer was gradually decreased to its set point at a rate of 0.11° C./min. This gradual change in temperature provides a lower supersaturation, thus avoiding primary nucleation during the start-up. This strategy avoids the longer period of start-up, necessary for washing of the primary nuclei (generated by step change in supersaturation) from column, which generally was a much longer process. Afterward, the column was run in “batch mode” to generate enough AAP nuclei on the D-mannitol, which again help in reaching the steady state faster. After ensuring a low supersaturation in the column, the feed and the discharge pumps were started. The pumps were operated with 510 s as an off time and 15 s as an on time at a flow rate of 280 ml/min. The high flow rate in pulses ensures efficient pumping of the slurry into and outside the column. The column was operated for enough time, to ensure a steady concentration of the slurry. FIG. 3 shows the concentration of acetaminophen measured using FTIR.

Analysis of Process Parameters and Product:

The online monitoring system, described above, helped in monitoring different process parameters. The same online monitoring system helped in ensuring a controlled operation of the crystallizer. To ensure accuracy of online monitoring tools, the slurry was filtered and the solution was analyzed offline for its concentration (using HPLC and density measurements). The product was collected on a filter paper, crystals were vacuum dried, and analyzed for their composition, size, structure, etc. XPRD, DSC, and optical microscopy were used for the analysis of product crystals. The product crystals were compressed into tablets using a tablet press.

Results and Discussion

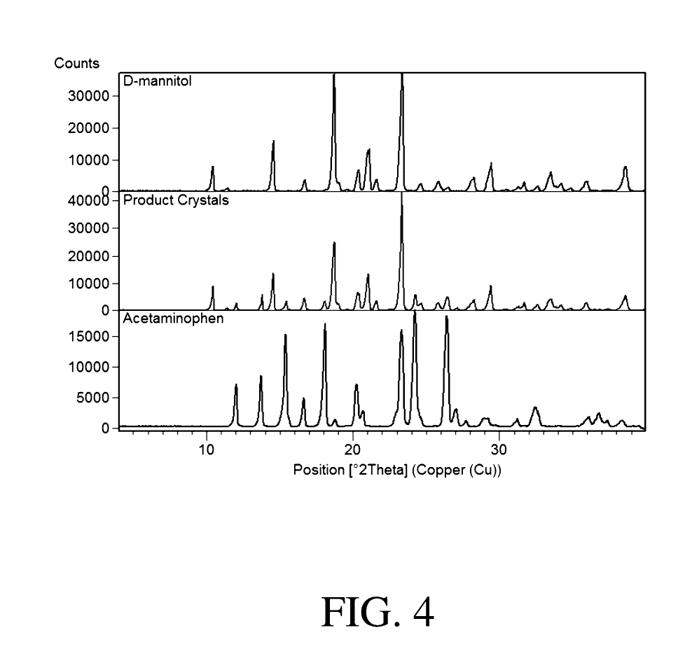

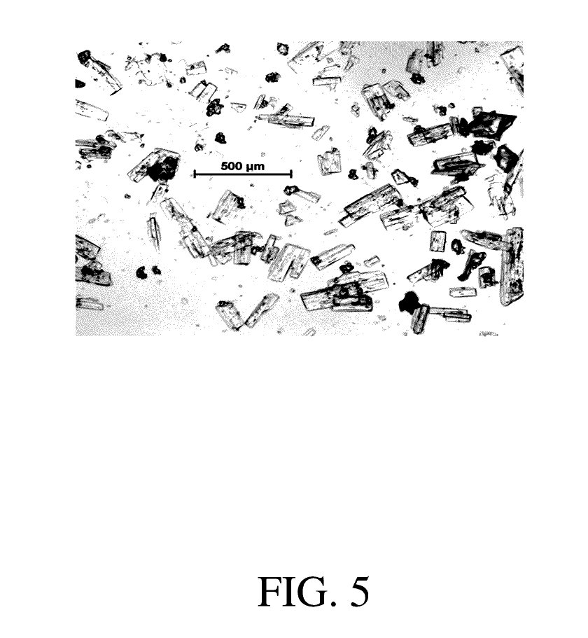

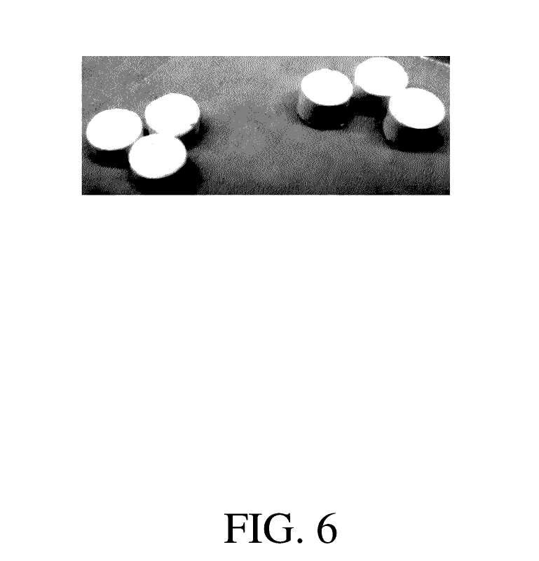

As shown in FIG. 3, the column was initially operated “batch wise” for the first ˜1000 minutes, and then the steady state or continuous operation was carried out. The column was operated at steady state for about 400 min. The product was analyzed using different techniques. FIG. 4 shows the XPRD results of the product, which ensured the presence of both D-mannitol and acetaminophen that were used as the raw material. Optical microscopy results, shown in FIG. 5, show the presence of heterogeneous nucleation (the acetaminophen was attached to the D-mannitol crystal surface). The tablets made out of product crystals were shown in the FIG. 6.

### Example 2

This example describes heterogeneous crystallization in a crystallizer.

Materials:

There were three main chemical components used in this example. The chemical system consisted of an API, excipient, and solvent. The API used in this example was Acetaminophen (Sigma-Aldrich), the excipient was D-Mannitol (Sigma-Aldrich), and the solvent was Ethanol (200 proof KOPTEC). The API-Excipient-Solvent selection was very important. The solvent was chosen such that the API was soluble in it, but the excipient was not. D-Mannitol as received by the manufacturer was not suitable. In order to control the crystal size distribution and improve the surface quality of the D-Mannitol used during experimentation of the fluidized bed crystallizer, a temperature cycling method of D-Mannitol in ethanol & deionized water mixture was performed. By increasing and decreasing the temperature of solution in a periodic manner, it leads to the dissolution of smaller particles and the subsequent growth of bigger particles. Temperature cycled particles were held at 43° C. for one hour in a 23.3 wt. % mixture of ethanol in water. The temperature was decreased to 20° C. in one hour and 20 minutes. The solution was then held at 20° C. for an hour. Pumping and filtration then started (the solution was maintained at 20° C. throughout the collection process). After the temperature cycling process, the D-Mannitol particles become larger with higher quality faces. For the actual continuous experimental runs, a solution of acetaminophen in ethanol was prepared by dissolving 196 mg of acetaminophen per 1 gram of ethanol.

### Experimental Setup

Process Description:

The experimental set up consists of a 4-liter glass jacketed feed vessel (Chemglass) equipped with an overhead stirrer (Heidolph, R Z R 2052 control), a 76.2 cm long and 25 mm inner diameter custom designed jacketed glass column (Ace Glass Incorporated), inlet pump (pump: Thermo Scientific, 1300-3140 & pump head: Thermo Scientific, 955-0000), outlet pump (pump: Thermo Scientific, 1300-3140 & pump head: Thermo Scientific, 955-0000), recirculation pump (pump: Cole Parmer, 7523-80 & pump head: Cole Parmer, 77200-62), heat exchangers used for temperature control of the feed vessel (Lauda, Proline RP845), glass column (Julabo, F32), and outlet line (Julabo, F32). The recirculation line was outfitted with an FTIR (Thermo Scientific, Nicolet 6700) that was equipped with a universal immersion probe (Axiom Analytical Incorporated, Dipper-210) operated by Omnic software. Additional equipment included a computer set up (Dell, Optiplex 745) with LabView software which received and sent data using a NI CompactDAQ chassis (National Instruments, NI cDAQ-9178) equipped with various signal modules, as well as RTD's and thermocouples for temperature control and system monitoring respectively.

The solution of acetaminophen in ethanol started off fully dissolved in a temperature controlled feed vessel where pre-processed D-mannitol was suspended with the use of an overhead mixer. The slurry was pumped into the bottom of the fluidized bed crystallizer. The material was internally entrained in a recirculation loop run at a high flowrate during the experiment. The addition of the recirculation loop was used to increase mass transfer and ensure a uniform suspension. The recirculation loop includes an FTIR probe which collects the data which was correlated to the concentration of solution and therefore provides an online concentration measurement. The column itself was temperature controlled. The slurry was pumped out of the column and into a vacuum filtration. The product was obtained from the filter plate and then dried for analytical testing.

### Experimental Description

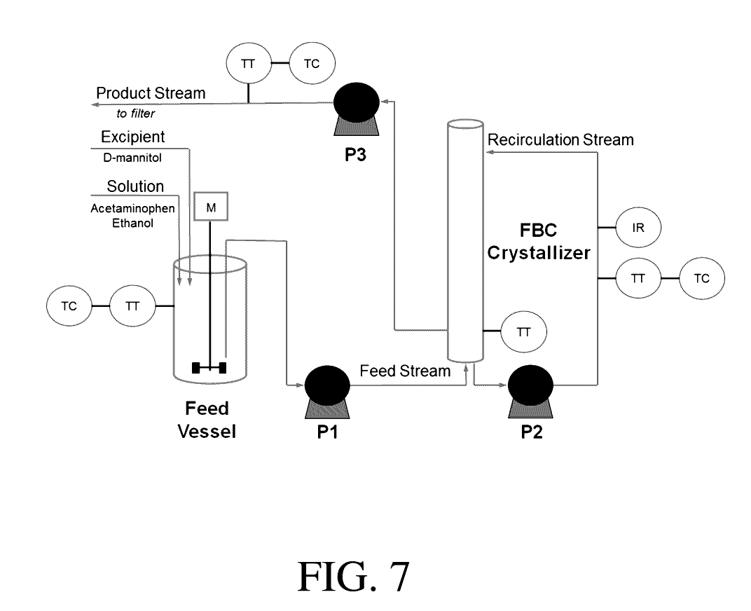

The crystallizer shown in FIG. 7 was used in this example.

Batch-Mode Startup Procedure:

The solution consisting of dissolved acetaminophen in ethanol was added to a jacketed feed vessel held at 25° C. For every 100 g of solution added to the feed vessel 8 g of D-Mannitol was also added. The D-Mannitol was held in suspension in the feed vessel via an overhead mixer run at 180 rpm. The slurry was added to the jacketed glass column crystallizer with the use of a peristaltic pump (P1) shown in FIG. 7. As the slurry was being added to the crystallizer the recirculation line pump (P2) was also started at 500 ml/min. Once the complete dead volume of the crystallizer and recirculation line was full, P1 was turned off. P2 remains on and continues to pump material around the column. The column temperature was reduced to the set point, 15° C. from 25° C., at a rate of 0.11° C./min. The contents of the column remained pumping until chemical equilibrium was reached. The gradual change in temperature provided for a lower supersaturation in order to avoid primary nucleation.

Continuous Run:

The inlet pump (P1) and outlet pump (P3) were turned on once the concentration of solution in the column has flat-lined as seen from the real-time concentration reading. The pumps were operated periodically, in that they turn on and off. They operate with an off time of 510 seconds and an on time of 15 seconds at a flow rate of 280 ml/min. The high flow rate in pulses ensured efficient pumping of the slurry into and outside the column without particles settling in the lines and clogging flow. This pumping pattern allows for low nominal flow rates 8 ml/min while maintaining the ability to pump dense slurries. The column was operated for enough time to ensure a steady concentration of the slurry recirculating in the column. Samples were collected during the steady state period at the outlet of the column. The concentration of solution was determined via a density meter calibration correlating density and temperature to concentration. The drug load was determined by dissolving the acetaminophen from the product in a known quantity of ethanol, filtering to obtain clear solution, and then analyzing the resultant solution via the same density meter technique. Microscope images of the product were also taken after experimentation to determine qualitatively if there was any primary or secondary nucleation occurring in the column during experimentation.

### Data Analysis

Sample Collection:

Samples were collected as the solution exits the column when P3 turns on. The solution was sent to a vacuum filtration for about 12 seconds. For the remaining 3 seconds the liquid slurry was collected. The slurry was filtered immediately to avoid any acetaminophen dissolving off the product crystals as the solution increases to room temperature. The solid state samples from the filter plate and the liquid samples were both analyzed.

Density Meter Analysis:

An offline density meter (Anton Paar, DMA 4100 M) was used to analyze the solid state samples and liquid samples obtained during experimentation. A calibration model relating density and temperature to concentration was determined prior to the experimental runs. The liquid samples were run immediately on the density meter at a specific temperature. The temperature at which the sample was run at, and the density reading given by the density meter was both fed into a model and the concentration of the liquid sample was determined. The solid state sample was added in a fixed quantity to a fixed quantity of ethanol. The acetaminophen on the product crystals was allowed to dissolve off the composite product crystals (Acetaminophen and D-Mannitol). The slurry was filtered and the clear solution was run on the density meter to obtain the concentration. The drug load was then back calculated from this measurement.

Microscope Image Analysis:

Optical microscope images of the solid product were taken to assess the degree of primary and secondary nucleation.

FTIR:

An online concentration measurement was obtained via an FTIR coupled with a chemometric model.

### Results and Discussion

Two continuous runs were performed. The concentration of the feed stream used 196 mg Acetaminophen/1 g of Ethanol in each case. There were two steady state temperatures for each run. Once the concentration of the solution “flat-lined”, the temperature of the column was changed to 12° C. and allowed to come to chemical equilibrium once again. Different size excipient seeds were used in each run.

TABLE 1
Continuous Run Experimental Summary

| | Starting | Ending | Ending | Excipient |
|---|---|---|---|---|
| | Concentration | Temperature 1 | Temperature 2 | Seed Size |
| Run # | (mg Ace/g EtoH) | (° C.) | (° C.) | (m²/g) |
| #1 | 196 | 15 | 12 | 0.0343 |
| #2 | 196 | 15 | 12 | 0.0976 |

TABLE 2
Experimental Results from Continuous Runs

| | | Starting | Steady State | |
|---|---|---|---|---|
| | Ending | Concentration | Concentration | |
| | Temperature | (mg Ace/ | (mg Ace/ | Steady State |
| Run # | (° C.) | g EtOH) | g EtOH) | Supersaturation |
| 1 | 15 | 196.4 | 176.2 | 0.024 |
| 1 | 12 | 196.4 | 166.6 | 0.028 |
| 2 | 15 | 196.5 | 175.8 | 0.021 |
| 2 | 12 | 196.5 | 169.8 | 0.047 |

TABLE 3
Drug Loading and Mass Balance Analysis

| | | Drug Loading | Drug Loading | |
|---|---|---|---|---|
| | Ending | From | From Solid | |
| Run | Temperature | Concentration | State Samples | DLsss/ |
| # | (° C.) | Profile (DLcp) (%) | (DLsss) (%) | DLcp * 100 |
| 1 | 15 | 17.4 | 19.9 | 114.4 |
| 1 | 12 | 23.5 | 21.6 | 91.9 |
| 2 | 15 | 17.8 | 20.9 | 117.6 |
| 2 | 12 | 21.7 | 23.6 | 108.9 |

### Conclusion

In this example, the design and initial experiments of a novel continuous crystallization process were presented. The crystallization process utilized the principles of heterogeneous nucleation in a fluidized bed system configuration whereby acetaminophen (API) nucleated on and grew on D-Mannitol (excipient). The fluidized bed system was run continuously and successfully produced composite particles of acetaminophen and D-Mannitol. The product crystals were filtered, and dried, and then directly compressed into tablets. In order to increase the strength of the tablets, additives were added to the product crystals manually and the resultant material was compressed into tablets. The addition of magnesium stearate (MgSt) to the product improved the friability of the tablet.

The surface area effect of the different D-Mannitol particles did not influence the drug load significantly. Upon further study, the crystal size distribution of the final crystals and the polymorph of the API could be controlled depending on the size and type of excipient used.

### Example 3

This example describes a continuous heterogeneous crystallization process in a fluidized bed crystallizer in which the active pharmaceutical ingredient, acetaminophen, was crystallized directly on the surface of an excipient, D-mannitol, within the crystallizer. The product was then filtered, dried, and compressed into tablets without the need for complex downstream processing steps such as milling, sieving, granulation, and blending. The crystallizer configuration was operated without significant formation of bulk API particles. Drug loads as high as 23.5% with residence times of approximately 86 minutes were achieved. The product was successfully compressed directly into tablets with a tensile strength of 0.61 MPa.

The pharmaceutical industry, with its current batch-wise approach to manufacturing, is faced with different challenges in the cost, reliability, and sustainability of their processes. There is a need for more efficient processing methods in the pharmaceutical industry. In some cases, it is expensive and time consuming to discover, develop, and launch new pharmaceuticals. The rising costs in the research and development of pharmaceuticals may far outpace the number of new chemical entities introduced to the market. In addition, there may be problems with batch processes in general, including poor yields, batch to batch variation, and the challenges, time, and expenses associated with scale up.

There are advantages in shifting from the traditional batch-mode manufacture of pharmaceuticals to a continuous-mode production strategy. The continuous manufacture of pharmaceuticals has the potential to reduce batch to batch variation, the amount of out-of-spec material needed to be rejected, ecological foot print, time to market due to easier scale up, and a variety of associated costs inherent in producing a drug and selling it in the marketplace.

Approximately 90% of all active pharmaceutical ingredients (APIs) are crystalline. Crystallization is necessary to separate and purify the API as well as to obtain the desired polymorph, shape, and crystal size distribution (CSD). The research and development of continuous crystallization processes which solve practical problems is therefore of great interest.

Some conventional pharmaceutical manufacturing process, the API molecules are crystallized, filtered, and then dried. The crystals typically encounter solid state processing steps after drying like milling, sieving, and dry/wet granulation before they are blended with excipients and other additives and compressed into tablets.

In this example, a continuous crystallization technology designed to streamline solid state downstream processing is described. In this crystallization process an API nucleates and grows directly on an excipient surface in a process known as heteroepitaxy. The product is therefore a stream of composite particles. In heteroepitaxy, crystals nucleate and grow on a crystalline substrate otherwise known as the heterosurface. The heterosurface orders prenucleation aggregates so nucleation becomes energetically favorable. In this crystallization process, the API (acetaminophen) nucleates and grows on an excipient surface (D-mannitol). The D-mannitol has been shown to induce faster induction times compared to other substrates. An important criterion in solvent selection is that the solvent has to be able to dissolve the API, but not the substrate.

Unless as otherwise indicated, the experimental setup of the crystallizer, methods, and material from Example 2 were used.

For heterogeneous crystallization of one substance on another (e.g. API on an excipient), any crystallization, either primary or secondary, other than that occurring on the excipient surface is referred to as “bulk nucleation” in this example and needs to be avoided. Bulk nucleation would negate the advantages of heterogeneous crystallization. A complicated separation process to remove the pure API crystals from the composite particles would otherwise have to be designed and implemented. These pure API crystals would then have to be recycled increasing the complexity of the overall process. The reduction and/or elimination of bulk nucleation are therefore an important aspect of this work. In order to achieve this goal, a modified FBC was designed and studied and is the subject of this example.

Approximately 90% of newly formed particles in some conventional industrial crystallizer are the result of secondary nucleation. A FBC configuration was selected because of its ability to suppress secondary nucleation. Secondary nucleation is effectively eliminated because of the absence of an impeller and the near plug flow pattern of the solution which reduced contact among crystals and the crystallizer wall and therefore the generation of secondary nuclei.

In some conventional FBC, a supersaturated solution in the metastable zone flows through a bed of particles and releases its supersaturation on them and the crystals grow as a result. The supersaturation is generally kept low to prevent primary nucleation. The large growing particles remain in the bed and are not circulated with the mother liquor and smaller particles. When the crystals in the bed grow to a desired size they are withdrawn from the crystallizer.

The FBC used in this example, like a traditional FBC, did not use an impeller. It was operated differently however. Particles were entrained in a suspension and continuously recirculated with the mother liquor at a relatively high flowrate during operation. This was done to increase mass transfer and ensure a uniform particle suspension. Product withdrawal was not dependent on size either and therefore the FBC was more similar to an MSMPR crystallizer in this respect compared to a traditional FBC.

A batch-wise FBC using standard equipment was set up prior to designing and manufacturing a custom continuous FBC. The batch-wise approach showed that bulk nucleation could be avoided and good amounts of growth were possible. These promising results led to the design of a continuous FBC.

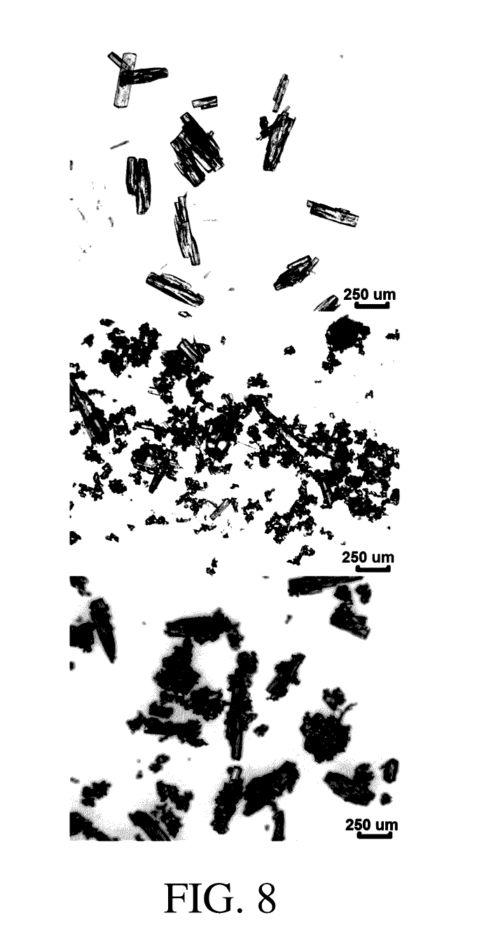

Short continuous crystallization trial runs in the continuous FBC were performed after the process flow of the system had been studied and optimized. High supersaturations led to bulk nucleation whereas lower supersaturations allowed for good amounts of growth on the D-mannitol substrates with very little bulk nucleation. FIG. 8 shows optical microscope images. The image at the top in FIG. 8 is a D-mannitol seeds. The image in the middle of FIG. 4 shows the composition at the 1 hour and 55 minute mark in one of the short continuous runs. There were a lot of acetaminophen particles that had not grown on the D-mannitol substrate surfaces. The starting concentration was 205.6 mg acetaminophen per 1 gram of ethanol. The ending temperature was 19.1° C. The image on the bottom shows the composition at the 5 hour mark using a lower supersaturation. Bulk nucleation was reduced significantly. The starting concentration was 196.3 mg acetaminophen per gram of ethanol. The ending temperature was 19.1° C.

Following these initial experiments, an extended continuous crystallization was run. Two important experiments were run. The starting concentration in each run was 196 mg acetaminophen per 1 gram of ethanol. There were two ending temperature set points, 15° C. and 12° C. An 8% suspension density was used in each run. Different sizes of excipient seeds were used in each run to determine if the surface area present had an effect on the final drug loading as shown in Table 4. It was hypothesized that the higher surface area of D-mannitol particles would contribute to more growth in the final product. Due to the specific method of production the surface quality of the two sets of seeds also differed.

TABLE 4
Seed Surface Area

| | Excipient Seed |
|---|---|
| Run | Size (m²/g) |
| 1 | 0.0343 |
| 2 | 0.0976 |

The operation of the FBC occurred in two modes. The first mode was the start-up mode where the solution in the FBC was brought to equilibrium. The second mode was where P1 and P3 were turned on and the continuous run began. This two-mode experimental procedure was used to reach steady state faster when the continuous run started. Both the first mode and the second mode of the experiment were clearly distinguished by their concentration profile.

In the start-up mode, only the recirculation pump, P2, was running and the temperature of the FBC was reduced to the first set point. During the start-up mode, there were three distinct periods. The first was when nucleation of the acetaminophen occurred on the D-mannitol substrates. The concentration in this period of time changed very little. The next period was when growth happens. This was categorized by a rapid decrease in concentration. The last period was the equilibrium period when the concentration in the FBC remained the same. It should be noted that this part of the experiment took a long time due to running the start-up mode overnight due to time constraints in operator shifts. The overall experimental time would be much shorter if this was not the case.

The start-up mode was followed by the continuous mode when the addition of feed solution and the removal of product were started by activating P1 and P3. When fresh feed entered the FBC the concentration began to rise and eventually hit a steady state concentration. To assess a different operating condition the temperature of the FBC was lowered to a different set point and the concentration in the FBC reached a second steady state.

Scatter in the concentration in the first and second steady state periods was due to the method of operation of the inlet and discharge pumps (P1 and P3). The pumps, P1 and P3, were run in bursts during the continuous stage to prevent particles from settling in the lines causing clogging issues while maintaining the ability to run at low nominal flowrates (8 ml/min). Zooming in on the concentration during this stage showed how the periodic pumping pattern affected the concentration in the FBC. The concentration increased as fresh feed was introduced to the FBC and decreased as crystallization occurred. For a scaled-up version of the FBC, higher flowrates and larger piping would minimize settling issues and obviate the need for periodic pump operation.

Liquid and solid state samples were taken periodically during the experimental runs. The steady state concentration of the liquid samples was averaged and used to calculate steady state supersaturation and theoretical drug loadings. The steady state concentration, supersaturation values, and drug loadings are given in Table 5. The solid state samples were dried and then tested for drug loading, bulk nucleation under the microscope, and were directly compressed into tablets whose strength was tested. The drug loadings were higher (about 1-3%) in the case of the solid state sample analysis (not shown) compared to the ones calculated from the steady state concentration. This was most likely due to evaporation and subsequent crystallization during filtration.

TABLE 5
Summary of Runs

| | | | | Drug | |
|---|---|---|---|---|---|
| | Ending | Steady State | Steady | Loading from | Resi- |
| | Temper- | Concentration | State | Concentration | dence |
| | ature | (mg Ace/ | Super- | Profile | Time |
| Run | (° C.) | g EtOH) | saturation | (%) | (min) |
| 1 | 15 | 176.2 | 0.024 | 17.4 | 86 |
| 1 | 12 | 166.6 | 0.028 | 23.5 | 86 |
| 2 | 15 | 175.8 | 0.021 | 17.8 | 84 |
| 2 | 12 | 169.8 | 0.047 | 21.7 | 84 |

The operating conditions were chosen to avoid primary nucleation, which was qualitatively assessed via optical microscopy. The FBC was run at low supersaturations. In addition, temperature changes in the FBC were done in a ramping manner as opposed to a crash cooling. In general, the vast majority of the growth appeared to have taken place on the D-mannitol substrates. There are small amounts of fine crystals occasionally seen and were most likely due to breakage during particle handling after filtration.

After the solid samples were dried, they were directly compressed into tablets without any pre-processing steps. This made for a very efficient pharmaceutical production process. It was observed that the samples produced nicely formed tablets reliably and were indeed directly compressible. These tablets were measured to have up to a 0.61 MPa tensile strength (see Table 6).

TABLE 6
Tensile strength measurements for Run #1 compressed tablets

| Tablet | Tensile Strength |
|---|---|
| Weight (mg) | (MPa) |
| 94.8 | 0.42 |
| 73.5 | 0.61 |

XRPD, Raman spectroscopy, and DSC were used to characterize the solid state product. XRPD and Raman were done to determine whether or not the product material was a composite of acetaminophen and D-mannitol. In the XRPD image, it was observed that characteristic peaks of both acetaminophen and D-mannitol were present, crystallinity was maintained, and no new polymorphs have formed. For the Raman technique, randomly selected sites on the same product crystal were selected for analysis. The Raman spectra showed peaks corresponding to both acetaminophen and D-mannitol. DSC was done to determine whether or not there was a significant change in melting point between the product crystals obtained compared to a physical mixture of acetaminophen and D-mannitol. The products from the two different runs agreed well in terms of melting point, but were slightly higher than the physical mixture.

In this example, the design of a continuous crystallization process is presented, in addition to the results of its operation. The crystallization process utilized the principles of heterogeneous nucleation in a customized fluidized bed system configuration whereby acetaminophen (API) nucleated and grew on D-mannitol (excipient). The fluidized bed system was run continuously and successfully produced composite particles of acetaminophen and D-mannitol with minimal bulk nucleation as observed with microscope images. The composite particles had a drug loading as high as 23.5% and were produced with an approximate residence time of 86 minutes. The drug loading could be increased if higher residence times were used. The product crystals were filtered, dried, and then directly compressed into tablets with a tensile strength of 0.61 MPa. Milling, granulation, and blending steps were all avoided before tablets were made.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto; the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. A method, comprising:
flowing a suspension comprising an excipient and a dissolved pharmaceutically active agent; and
while the suspension is flowing, crystallizing at least a portion of the pharmaceutically active agent on at least a portion of a surface of the excipient.

2. A method, comprising:
crystallizing a pharmaceutically active agent in a suspension comprising the pharmaceutically active agent, an excipient, and a solvent to form a plurality of pharmaceutically active agent crystals, wherein greater than or equal to about 80% of the pharmaceutically active agent crystals are in contact with a surface of the excipient.

3. (canceled)

4. (canceled)

5. A method, comprising:
crystallizing a pharmaceutically active agent in a suspension comprising the pharmaceutically active agent, an excipient, and a solvent to form a plurality of pharmaceutically active agent crystals, wherein fewer than to about 20% of the pharmaceutically active agent crystals are formed via a bulk crystallization process and/or secondary nucleation process.

6. The method of claim 1, wherein flowing the suspension comprises flowing the suspension in a continuous flow crystallizer.

7. The method of claim 1, further comprising forming a pharmaceutical product comprising the pharmaceutically active agent and the excipient.

8. The method of claim 7, wherein the pharmaceutical product is a pharmaceutically acceptable product.

9. The method of claim 1, wherein the concentration of the pharmaceutically active agent in the suspension is in a metastable zone of supersaturation.

10. The method of claim 1, wherein the excipient is a solid.

11. The method of claim 1, wherein the concentration of dissolved excipient in the suspension is less than or equal to about 1 mg/L.

12. The method of claim 1, wherein the suspension is in a chamber and wherein the volume of the suspension in the chamber is greater than or equal to about 100 mL.

13. The method of claim 5, wherein less than or equal to about 10% of the crystallization is bulk crystallization.

14. The method of claim 2, wherein greater than or equal to about 90% of the crystallization occurs on at least a portion of a surface of the excipient.

15. The method of claim 1, wherein crystallizing comprises nucleation and crystal growth in the presence of a solid excipient.

16. (canceled)

17. The method of claim 1, further comprising filtering the at least a portion of the suspension comprising the pharmaceutically active agent crystals.

18. The method of claim 1, wherein the energy per volume absorbed by the pharmaceutically active crystal as a result of a collision does not exceed the toughness of the pharmaceutically active crystals.

19. The method of claim 1, wherein the excipient comprises a plurality of particles.

20. The method of claim 1, further comprising forming a pharmaceutically acceptable tablet comprising the pharmaceutically active agent crystals.

21. The method of claim 5, further comprising separating the pharmaceutically active agent crystals formed via bulk crystallization from the pharmaceutically active agent crystals formed via heterogeneous crystallization.

22. The method of claim 5, comprising dissolving pharmaceutically active agent crystals formed via bulk crystallization to form dissolved pharmaceutically active agent and recycling the dissolved pharmaceutically active agent.
